(19) United States
(12) Patent Application Publication
(10) Pub. No.: US 2025/0026686 A1
(43) Pub. Date: Jan. 23, 2025

(54) MICROBIALLY INDUCED CARBONATE PRECIPITATION-BASED VERTICAL BARRIER AND ITS PREPARATION METHOD

(71) Applicant: Southeast University, Jiangsu (CN)

(72) Inventors: Ningjun Jiang, Jiangsu (CN); Yu Zhang, Jiangsu (CN); Shiqi Liu, Jiangsu (CN); Xiangrui Xu, Jiangsu (CN)

(73) Assignee: Southeast University, Jiangsu (CN)

(21) Appl. No.: 18/713,190

(22) PCT Filed: Dec. 29, 2022
(86) PCT No.: PCT/CN2022/143184

§ 371 Date: May 24, 2024

(30) Foreign Application Priority Data
Oct. 21, 2022 (CN) 202211291419.8

(51) Int. Cl. C04B 24/00; C04B 14/10; C04B 28/00; C04B 103/00; C12N 1/20; C12R 1/00

(52) U.S. Cl. CPC C04B 24/00; C04B 14/104; C04B 28/00; C12N 1/20; C04B 2103/0001; C12R 2001/00

(57) ABSTRACT

The present invention discloses a microbially induced carbonate precipitation (MICP)-based vertical barrier, and a preparation method thereof, wherein a raw material of the vertical barrier includes in-situ soil, Ca-bentonite, a bacteria-containing culture medium, and a cementing solution. The bacteria-containing culture medium comprises flocculent ureolytic bacteria flora. The proportions of dry the Ca-bentonite to in-situ soil, the bacterial-containing culture medium to the cementing solution, and the cementing solution to the Ca-bentonite are specified to be 1:2, (2˜5):1, and (1˜2.5):10, respectively, to ensure optimal performance. The present invention addresses challenges related to the insufficient impermeability of Ca-bentonite-based barriers in the application of vertical barrier, and aims to reduce both the cost and carbon emissions associated with their construction.

### TECHNICAL FIELD

This invention relates to the field of pollution barrier. It specifically involves a vertical barrier utilizing the technology of microbially induced carbonate precipitation and describes a method for its preparation.

### DESCRIPTION OF RELATED ART

In the field of managing and controlling risks associated with soil and groundwater pollution, vertical barriers have emerged as highly effective and practical solutions. They achieve contamination containment by isolating polluted groundwater from clean regions, thanks to their low permeability coefficients. The adoption of soil-bentonite backfill in constructing these barriers is noted for its cost-effectiveness, relying primarily on bentonite as the barrier material, leading to its widespread acceptance and further development worldwide. However, in China, due to historical and technical reasons, the preference leans towards using cement-mixed soil for constructing these barriers. Despite the enhanced strength of soil-cement barriers over those made from soil-bentonite or a combination of soil, cement, and bentonite, they often fall short in terms of permeability. The construction of pure cement barriers introduces additional difficulties such as heightened costs and diminished permeability due to erosion by contaminants, undermining their preventive capabilities against contamination. Therefore, it is very necessary to vigorously develop the bentonite-based vertical barriers.

Generally speaking, bentonite, the core component of bentonite-based vertical barriers, is categorized into two types, namely calcium (Ca)-bentonite and sodium (Na)-bentonite types. Among them, the properties of Na-bentonite are far better than those of Ca-bentonite, and thus, Na-bentonite is generally used in the field of pollution barrier. However, around the world, Ca-bentonite is more widely distributed than Na-bentonite. In countries like China, the production of Na-bentonite is relatively low compared to Ca-bentonite, leading to the prevalent use of Na-enhanced Ca-bentonite. Despite this modification, the impermeability achieved does not equal that of natural Na-bentonite, and costs are driven higher. Therefore, optimizing the use of Ca-bentonite stands out as a significant challenge.

Lately, microbially induced carbonate precipitation (MICP) technology refers to a process of using certain types of bacteria in nature whose metabolism can produce ureolytic bacteria to catalyze urea hydrolysis, and combining of carbonate ions and ammonium ions produced after the hydrolysis of urea with free metal cations to form gelled crystals. The application of MICP technology requires low production energy consumption and production costs, and can also reduce greenhouse gas emissions. Generally, in existing research, most of the metal cations used are calcium ions, which form calcium carbonate precipitates after combining with carbonate ions. On the one hand, it enables cementing the rock and soil particles, and on the other hand, it enables filling the pores in the soil, and at the same time, achieving the effects of reduced permeability coefficient and increased of strength. Therefore, based on such characteristics, applying MICP to improve the performance of Ca-bentonite vertical barriers appears both feasible and promising.

### SUMMARY OF THE INVENTION

Technical problem to be solved: To overcome the identified technical issues, this invention presents a vertical barrier based on microbially induced carbonate precipitation, along with a method for its preparation. It seeks to solve the problem of poor hydraulic conductivity found in Ca-bentonite in vertical barrier application, aiming to reduce both the cost and carbon footprint of its manufacturing process.

Technical solution: A microbially induced carbonate precipitation-based vertical barrier, in which a raw material of the vertical barrier includes in-situ soil, Ca-bentonite, a bacteria-containing nutrient solution, and a cementing solution, wherein the bacteria-containing nutrient solution is a nutrient solution includes flocculent ureolytic bacteria flora. A dry mass of the Ca-bentonite to a mass of the in-situ soil is between 5% and 15%, a mass ratio of the bacteria-containing solution to the cementing solution is 1:2, a mass ratio of the cementing solution to the Ca-bentonite is between 2:1 and 5:1, and a mass ratio of the bacteria-containing nutrient solution to the in-situ soil is between 1:10 and 2.5:10.

Preferably, the ureolytic bacteria is Sporosarcina pasteurii.

Preferably, the bacteria-containing nutrient solution is prepared by inoculating a stock solution of the ureolytic bacteria into a nutrient solution at a 1% volume ratio, and incubated for 24 hours with agitation.

Preferably, the cementing solution contains urea and sodium salts, with the molar concentration ratio of the urea to the sodium salts ranging from 1:1 to 5:1.

Preferably, the concentration of the cementing solution ranges from 1 mol/L to 5 mol/L.

A method for preparing the microbially induced carbonate precipitation-based vertical barrier includes the following steps:
step (1), mixing the bacteria-containing nutrient solution with the in-situ soil, and allowing the mixture to cure;
step (2), incorporating the Ca-bentonite into the cementing solution and stirring to prepare a bentonite slurry; and
step (3), combining the bentonite slurry with the in-situ soil containing ureolytic bacteria and curing the mixture to obtain the microbially induced carbonate precipitation-based vertical barrier.

Preferably, a time of the curing for both the steps 1 and 3 should be at least 24 hours.

Preferably, a time of the mixing in the step 2 should be between 10 to 20 minutes.

Advantageous effect: This invention substitutes Na-bentonite, typically used in bentonite-based barriers, with Ca-bentonite, and modifies the Ca-bentonite through the ureolytic bacteria to reduce the permeability of these barriers. By introducing MICP technology and the application of Ca-bentonite technology, the present invention broadens the selection of material choices for constructing vertical barriers.

The present invention effectively reduces the cost of the vertical barrier: bentonite as the main material of the vertical barrier, Ca-bentonite is more than 70% cheaper than Na-bentonite. The present invention has the advantage of being low-carbon and environmentally friendly: the conventional production process of Na-modified bentonite requires heating of the bentonite and produces a large amount of waste liquid at the same time, and by using MICP technology to place the sodium activation process after the construction of the vertical barrier is completed, carbon emissions will be significantly reduced.

The application of MICP technology requires low production energy consumption and production costs, and can also reduce greenhouse gas emissions. The calcium ions of Ca-bentonite combine with carbonate ions to form calcium carbonate precipitation. On one hand, it enables cementing the rock and soil particles, and on the other hand, it enables filling the pores in the soil, and at the same time, achieving the effects of reduced permeability coefficient and increased of strength.

### BRIEF DESCRIPTION OF THE DRAWINGS

FIG. 1 provides a schematic illustration of the inventive process.

### DETAILED DESCRIPTION OF THE EMBODIMENTS

The invention is further elaborated upon with the help of drawings and specific examples.

### Embodiment 1

The raw material of the microbially induced carbonate precipitation-based vertical barrier includes in-situ soil, Ca-bentonite, a bacteria-containing nutrient solution, and a cementing solution, wherein the bacteria-containing nutrient solution is a nutrient solution comprises flocculent ureolytic bacteria flora, which is prepared by inoculating 1% of the stock solution of the ureolytic bacteria into a nutrient solution, and incubating for 24 hours. The mass ratio of the in-situ soil, the Ca-bentonite, the bacteria-containing nutrient solution and the cementing solution is 10:1:1:2.

The selected ureolytic bacteria are Sporosarcina pasteurii, in which the bacteria strain not only has an enhanced urease production, but also has a higher tolerance to heavy metal ions compared to other bacteria, making it ideal for contaminated environments.

The nutrient solution is used to provide microorganisms with essential nutrients for microbial growth and biomineralization, which includes 15 g/L of yeast extract, 10 g/L of ammonium sulfate, and 15.748 g/L of trihydroxymethylaminotoluene.

The bacteria-containing nutrient solution is prepared by inoculating the ureolytic bacteria into the nutrient solution, mixing and incubating with agitation for 24 hours.

The solutes of the cementing solution are urea and sodium salts, and a molar concentration ratio of the urea and the sodium salts is 1:1.

The concentration of the cementing solution is set at 1 mol/L/L.

A preparation method for the microbially induced carbonate precipitation-based vertical barrier includes the following steps:
step (1), mixing the bacteria-containing nutrient solution with the in-situ soil, and allowing the mixture to cure for not less than 24 hours;
step (2), adding the Ca-bentonite to the cementing solution and stirring to prepare a bentonite slurry; and
step (3), mixing the bentonite slurry with the in-situ soil, and curing for at least 24 hours to obtain the microbially induced carbonate precipitation-based vertical barriers.

In conventional MICP technology, bacteria are not transported uniformly through the soil and remain mainly on the surface of the soil being cured, resulting in nonuniform curing or sealing. Therefore, if the nutrient solution and the cementing solution are injected at the same time, it will cause the bacterial community to start blocking during the development process, thus affecting the uniformity problem.

The present invention uses two grouting injections: injecting the bacteria-containing nutrient solution and the cementing liquid into the in-situ soil respectively to separate the development of bacterial flora and the cementation process, which effectively solving the problem of poor uniformity caused by blocking of microorganisms during mineralization.

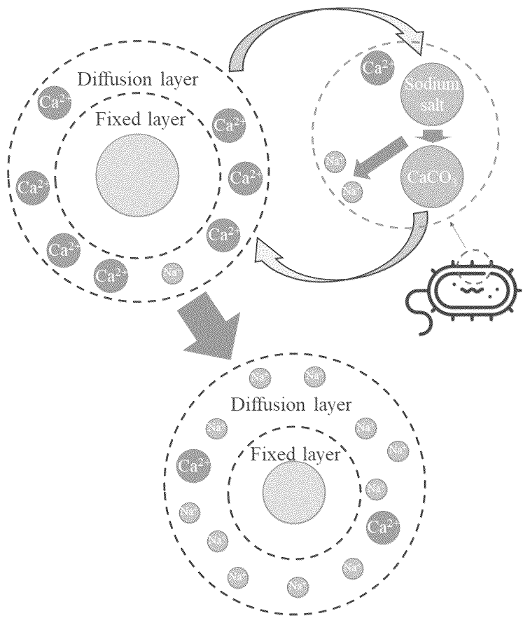

As illustrated in FIG. 1, the present invention replaces commonly used Na-bentonite or Na-modified bentonite in soil-bentonite vertical barriers with Ca-bentonite, and utilizes the characteristics of ureolytic bacteria to hydrolyze urea to produce carbonate, the calcium ions in Ca-bentonite are replaced by sodium ions through the mineralization of microorganisms. At the same time, ammonium ions are produced, which leads to the thickening of the diffusion layer and the increase of weakly bound water in the diffuse double layer of bentonite particles. The formed calcium carbonate precipitation fills the pores in the soil, thereby achieving the effect of reducing the permeability coefficient.

1. A MICP-based vertical barrier, wherein a raw material of the vertical barrier comprising in-situ soil, Ca-bentonite, a bacteria-containing nutrient solution, and a cementing solution,
wherein the bacteria-containing culture medium is inoculated with ureolytic bacteria, a dry mass of the Ca-bentonite is 5%˜15% of a mass of the in-situ soil, a mass ratio of the bacteria-containing culture medium to the cementing solution is 1:2, a mass ratio of the cementing solution to the Ca-bentonite is between 2:1 and 5:1, and a mass ratio of the bacteria-containing culture medium to the in-situ soil ratio is between 1:10 and 2.5:10.

2. The MICP-based vertical barrier according to claim 1, wherein the ureolytic bacteria is Sporosarcina pasteurii.

3. The MICP-based vertical barrier according to claim 1, wherein the bacteria-containing culture medium is prepared by inoculating a stock solution of the ureolytic bacteria into a culture medium at a volume ratio of 1%, and incubating for 24 hours with agitation.

4. The MICP-based vertical barrier according to claim 1, wherein solutes of the cementing solution are urea and sodium salts, and a molar concentration ratio of the urea to the sodium salts is ranging from 1:1 to 5:1.

5. The MICP-based vertical barrier according to claim 1, wherein a concentration of the cementing solution ranges from 1 mol/L to 5 mol/L.

6. A preparation method of the MICP-based vertical barrier according to claim 1, comprising the following steps:
step (1), mixing the bacteria-containing culture medium with the in-situ soil, and allowing the mixture to cure;
step (2), incorporating the Ca-bentonite into the cementing solution and stirring to prepare a bentonite slurry; and
step (3), mixing the bentonite slurry with the in-situ soil, and curing, to obtain the microbially induced carbonate precipitation-based vertical barrier.

7. The preparation method of the MICP-based vertical barrier according to claim 6, wherein a time of the curing for both the steps 1 and 3 is not less than 24 hours, and a time for the stirring in the step 2 is between 10 to 20 minutes.
